(19) United States
(12) Patent Application Publication
(10) Pub. No.: US 2019/0353613 A1
(43) Pub. Date: Nov. 21, 2019

(54) ELECTROPHEROGRAM ANALYSIS

(71) Applicant: IntegenX Inc., Pleasanton, CA (US)

(72) Inventors: David King, Menlo Park, CA (US); Bruce Goldman, Redwood City, CA (US)

(21) Appl. No.: 16/315,616

(22) PCT Filed: Dec. 8, 2017
(86) PCT No.: PCT/US2017/065447

§ 371 Date: Jan. 4, 2019

Related U.S. Application Data
(60) Provisional application No. 62/432,512, filed on Dec. 9, 2016.

(51) Int. Cl. G01N 27/447; G16B 45/00

(52) U.S. Cl. CPC G01N 27/44726; G16B 45/00

(57) ABSTRACT

Methods for analyzing raw electropherogram data are disclosed. Some methods includes extracting color data as a function of time or position from the raw electropherogram darta, selecting from the electropherogram one or more peaks that contain color data for a first dye and substantially no color data from other dyes used in electrophoresis. The method also includes determining the color spectrum of the first dye, and using the color spectrum of the first dye to deconvolve the color data of the raw electropherogram data to separate the contributions of each of the dyes to the raw electropherogram data. Systems and apparatus for producing electropherograms are also disclosed.

### CROSS-REFERENCE TO RELATED APPLICATIONS

This application claims benefit of U.S. Provisional Patent Application No. 62/432,512, filed Dec. 9, 2016, which is incorporated herein by reference in its entirety and for all purposes.

### BACKGROUND

Electrophoresis is the motion of dispersed particles relative to a fluid under the influence of a spatially uniform electric field. It may be caused by the presence of a charged interface between the particle surface and the surrounding fluid. Electrophoresis is the basis for a number of analytical techniques used in biochemistry for separating molecules by size, charge, or binding affinity. Electrophoresis and other separation technologies sometimes use two more dyes to distinguish two or more nucleic acid sequences or other features.

### SUMMARY

One aspect of this disclosure pertains to methods of producing an electropherogram from raw electropherogram data comprising a sequence of one or more peaks, each peak comprising signal intensity values as a function of wavelength and time or position, and each peak corresponding to one or more unique macromolecules, each macromolecule tagged with one of a plurality of different dyes. Each peak has a spectral contribution from one or more of the dyes. Such methods may be characterized by the following operations: (a) receiving the raw electropherogram data; (b) for a first dye from plurality of different dyes, selecting from the raw electropherogram data one or more color peaks that contain signal intensity versus wavelength data for the first dye and substantially no signal intensity for any other dyes of the plurality of different dyes; (c) determining, from the one or more color peaks identified in (b), a color spectrum of the first dye, wherein the color spectrum of the first dye comprises signal intensity values as a function of wavelength for only the first dye; and (d) using the color spectrum of the first dye, together with color spectra of the other dyes of the plurality of different dyes, to deconvolve the raw electropherogram data. The deconvolving may separate the contributions of each of the dyes to the raw electropherogram data and produce the electropherogram.

In some embodiments, a method repeats operations (b)-(c) for at least one of the other dyes of the plurality of different dyes. In other words, the first dye is replaced with a different one of the other dyes for each pass through (b)-(c). In some embodiments, a method repeats operations (b)-(c) for each of the different dyes.

In certain embodiments, the macromolecules are amplicons from amplification reactions of DNA sequences at two more loci of a genome or chromosome. In some cases, the genome is a human genome. In some cases, the loci are at polymorphism sites. In some cases, the polymorphism sites are STR sites. In some cases, there are at least about sixteen loci and/or at least three dyes. In certain embodiments, the methods additionally include using the electropherogram to identify alleles of an individual who originated a sample that produced the raw electropherogram data.

In certain embodiments, the method additionally includes performing electrophoresis on a sample including the macromolecules. In such embodiments, performing electrophoresis generates the raw electropherogram data. Note that method may additionally include one or more sample preparation operations prior to performing electrophoresis. Such operations may include, for example, obtaining a sample (e.g., a crime scene sample or a buccal sample), extracting cells from the sample, lysing cells, extracting nucleic acids from the sample, amplifying particular loci of the nucleic acids or a whole genome, etc.

In certain embodiments, the color data is provided in between fifty and five hundred distinct color channels (e.g., channels of spectrophotometer). In some cases, the signal intensity versus wavelength data for the color peaks was obtained using a spectrophotometer.

In certain embodiments, selecting one or more color peaks that contain signal intensity versus wavelength data for the first dye and substantially no signal intensity for any other dyes of the plurality of different dyes includes: applying criteria for selecting one or more substantially isolated and substantially spectrally pure color peaks from the raw electropherogram data. In some implementations, the criteria include identifying color peaks having a portion that increases or decreases monotonically in a wavelength dimension (the positions on the wavelength dimension represent distinct wavelengths). In some embodiments, the criteria include identifying color peaks having a portion that has a slope in a wavelength dimension of at least a predefined value. In some embodiments, the criteria include identifying peaks that are separated from other peaks by at least a threshold time duration or position difference.

In some implementations, applying the criteria for selecting one or more substantially isolated and substantially spectrally pure color peaks identifies multiple substantially isolated and substantially spectrally pure peaks. In some cases, the method additionally includes an operation of combining the spectra of the multiple substantially isolated and substantially spectrally pure color peaks to produce the color spectrum of the first dye. Combining the spectra the spectra of the multiple substantially isolated and substantially spectrally pure color peaks may include producing a weighted average of the spectra of the multiple substantially isolated and substantially spectrally pure color peaks. Producing the weighted average of the spectra of the multiple substantially isolated and substantially spectrally pure color peaks may include weighting each of the spectra of the substantially isolated and substantially spectrally pure color peak according to its peak height and/or its peak width.

In certain embodiments, the methods additionally include: (i) correlating multiple substantially isolated and substantially spectrally pure color peaks to identify a subset of said multiple peaks that are more highly correlated than other of said multiple peaks that are not in the subset; and (ii) combining the subset of substantially isolated and substantially spectrally pure peaks to produce the color spectrum of the first dye.

In certain embodiments, the methods additionally include preparing a calibration matrix from the color spectrum of the first dye the other dyes of the plurality of different dyes and the other dyes of the plurality of different dyes. In such embodiments, using the color spectrum of the first dye, together with color spectra of the other dyes of the plurality of different dyes to deconvolve the raw electropherogram data includes applying the calibration matrix to the raw electropherogram data. In some implementations, the calibration matrix includes color spectra of all the plurality of different dyes.

In some implementations, a single sample is employed to produce the raw electropherogram data and the one or more color peaks that contain signal intensity versus wavelength data for the first dye and substantially no signal intensity for any other dyes of the plurality of different dyes.

In certain embodiments, the macromolecules are oligonucleotides. In certain embodiments, the number of unique macromolecules producing the raw electropherogram data is greater than the number of different dyes tagging the unique macromolecules. In certain embodiments, the method additionally includes using the electropherogram to identify a macromolecule corresponding to a peak in the raw electropherogram data.

Another aspect of the disclosure pertains to systems that may be characterized by the following features: (a) a capillary tube arranged to receive a sample comprising a plurality of unique macromolecules and run the sample through the capillary tube so that different ones of the unique macromolecules pass through an interrogation region of the capillary tube at different times; (b) optical elements arranged with respect to one another to receive color signals from the interrogation region; and (c) a controller for performing an internal calibration on a dye. In certain embodiments, the controller is designed or configured to perform or cause to be performed: (i) converting the color signals into raw electropherogram data comprising a sequence of peaks, each peak comprising signal intensity values as a function of wavelength and time or position and each peak corresponding to one or more unique macromolecules, each macromolecule tagged with one of a plurality of different dyes, wherein each peak has a spectral contribution from one or more of the dyes, (ii) for a first dye from plurality of different dyes, selecting from the raw electropherogram data one or more color peaks that contain signal intensity versus wavelength data for the first dye and substantially no signal intensity for any other dyes of the plurality of different dyes, (iii) determining, from the one or more color peaks identified in (ii), a color spectrum of the first dye, wherein the color spectrum of the first dye comprises signal intensity values as a function of wavelength for only the first dye, and (iv) using the color spectrum of the first dye, together with color spectra of the other dyes of the plurality of different dyes, to deconvolve the raw electropherogram data to separate the contributions of each of the dyes to the raw electropherogram data and produce the electropherogram.

In certain embodiments, the controller is further designed or configured to perform or cause to be performed one or more of the above computational method operations. The controller may receive, store, or generate excutable program instruction for causing any of the recited method operations to be performed.

Another aspect of this disclosure pertains to methods of analyzing a sample comprising one or more unique macromolecules tagged with one of a plurality of different dyes. Such methods may be characterized by the following operations: (a) performing an electrophoresis run on the sample to produce first raw electropherogram data comprising a sequence of peaks, each corresponding to one or more of the unique macromolecules, wherein each peak has a spectral contribution from one or more of the plurality of different dyes; (b) analyzing the first raw electropherogram data and identifying an uncalibrated dye, from among the plurality of different dyes associated with the macromolecules, for which a substantially pure spectrum is not identified from the raw electropherogram data; (c) identifying a substantially pure spectrum of the uncalibrated dye from second raw electropherogram data of a related electrophoresis run; and (d) using the substantially pure spectrum of the uncalibrated dye, from the second raw electropherogram data, to deconvolve the first raw electropherogram data to separate the contributions of each of the plurality of different dyes to the first raw electropherogram data to thereby produce a first electropherogram.

In certain embodiments, the methods additionally include the following operation: from the first raw electropherogram data, extracting multi-channel color data as a function of time or position, where the color data represents the spectral contributions from the plurality of different dyes.

In certain embodiments, the related electrophoresis run is a next sequential electrophoresis run on the same apparatus as used to produce the first raw electropherogram data. In certain embodiments, the first raw electropherogram data and the second raw electropherogram data are produced using runs conducted at the same position in a single apparatus. In certain embodiments, the first raw electropherogram data and the second raw electropherogram data are produced using runs conducted at two different positions at the same time in a single apparatus.

In some cases, a method additionally includes, prior to deconvolving the first raw electropherogram data, scaling the substantially pure spectrum of the uncalibrated dye, from the second raw electropherogram data. The scaling may involve modifying the substantially pure spectrum of the uncalibrated dye using information obtained about the spectra of a first calibrated dye obtained using both the first raw electropherogram data and the second raw electropherogram data.

In certain embodiments, the number of unique macromolecules is greater than the number of different dyes. In certain embodiments, each peak of the first raw electropherogram data comprises signal intensity values as a function of wavelength and time or position.

Still another aspect of the disclosure pertains to systems that can be characterized by the following elements: (a) a capillary tube arranged to receive a sample comprising a plurality of unique macromolecules and run the sample through the capillary tube so that different ones of the unique macromolecules pass through an interrogation region of the capillary tube at different times; (b) optical elements arranged with respect to one another to receive color signals from the interrogation region; and (c) a controller that can produce of facilitate production of an electropherogram using a dye calibration spectrum obtained from a related electrophoresis run (related to the run for which calibration is performed). In certain embodiments, the controller is designed or configured to perform or cause to be performed: (i) converting the color signals into raw electropherogram data comprising a sequence of peaks, each corresponding to one or more of the plurality of unique macromolecules tagged with one of a plurality of different dyes, (ii) performing an electrophoresis run on the sample to produce first raw electropherogram data comprising a sequence of peaks, each corresponding to one or more of the unique macromolecules, wherein each peak has a spectral contribution from one or more of the plurality of different dyes, (iii) analyzing the first raw electropherogram data and identifying an uncalibrated dye, from among the plurality of different dyes associated with the macromolecules, for which a substantially pure spectrum is not identified from the raw electropherogram data, (iv) identifying a substantially pure spectrum of the uncalibrated dye from second raw electropherogram data of a related electrophoresis run, and (v) using the substantially pure spectrum of the uncalibrated dye, from the second raw electropherogram data, to deconvolve the first raw electropherogram data to separate the contributions of each of the plurality of different dyes to the first raw electropherogram data. This may produce or facilitate production of a first electropherogram.

In certain embodiments, the controller is further designed or configured to perform or cause to be performed one or more of the computational method operations of the preceding aspect of the disclosure. The controller may receive, store, or generate excutable program instruction for causing any of the recited method operations to be performed.

These and other features of the disclosure will be described in more detail below with reference to the associated drawings.

### BRIEF DESCRIPTION OF THE DRAWINGS

FIG. 1 presents a schematic illustration of apparatus configured to perform sample preparation (e.g., lysis, nucleic acid extraction, and nucleic acid amplification) followed by electrophoresis.

FIG. 2 presents a simplified example of matrix operations that may be employed to deconvolute raw electropherogram data.

FIG. 3 presents an example of raw electropherogram data that may be analyzed in accordance with certain methods disclosed herein.

FIG. 4 presents an example of an electropherogram that may be produced from raw electropherogram data in accordance with certain methods described herein.

FIG. 5 presents an example of raw electropherogram data that may be obtained when operating an electrophoresis optical system with long exposure times.

FIG. 6 presents, for comparison purposes, an example of raw electropherogram data that may be obtained when operating an electrophoresis optical system with short exposure times.

FIG. 7 is a process flow chart illustrating how long exposure scan data and short exposures scan data can be used together to provide improved raw electropherogram data.

FIG. 8 presents an example of grafted electropherogram data that may be produced using long exposure scan data and short exposures scan data.

FIG. 9 is a process flow diagram illustrating how spectrally pure calibration data may be obtained, in sample, from raw electropherogram data for dyes that generate the raw electropherogram data.

FIG. 10 is a process flow diagram depicting how spectrally pure calibration data can be obtained, out of sample, from raw electropherogram data for dyes that generate the raw electropherogram data.

FIG. 11 presents a schematic depiction of an analyte preparation module that may be used to prepare samples for electrophoresis in accordance with certain embodiments herein.

FIG. 12 presents a schematic illustration of an analysis module for a capillary electrophoresis system may be used in accordance with certain embodiments herein.

### DETAILED DESCRIPTION OF AN EMBODIMENT

### Introduction

While various embodiments of the invention are shown and described herein, those skilled in the art that such embodiments are provided by way of example only. Numerous variations, changes, and substitutions may occur to those skilled in the art without departing from the invention. It should be understood that various alternatives to the embodiments of the invention described herein may be employed.

The term “sample,” as used herein, refers to a sample containing biological material. A sample may be, e.g., a fluid sample (e.g., a blood sample) or a tissue sample (e.g., a cheek swab). A sample may be a portion of a larger sample. A sample can be a biological sample having a nucleic acid, such as deoxyribonucleic acid (DNA) or ribonucleic acid (RNA), or a protein. A sample can be a forensic sample or an environmental sample. A sample can be pre-processed before it is introduced to the system; the preprocessing can include extraction from a material that would not fit into the system, quantification of the amount of cells, DNA or other biopolymers or molecules, concentration of a sample, separation of cell types such as sperm from epithelial cells, concentration of DNA using, e.g., bead processing or other concentration methods or other manipulations of the sample. A sample can be carried in a carrier, such as a swab, a wipe, a sponge, a scraper, a piece punched out a material, a material on which a target analyte is splattered, a food sample, a liquid in which an analyte is dissolved, such as water, soda. A sample can be a direct biological sample such as a liquid such as blood, semen, saliva; or a solid such a solid tissue sample, flesh or bone.

The term “dye” is used to refer to any compound or composition that can be detected and classified by its electromagnetic spectrum. Often dyes emit, transmit, or absorb electromagnetic radiation in a particular narrow spectral band, which can be in the visible, infrared, ultraviolet, or other region of the electromagnetic spectrum. In the context of this disclosure, a dye can be associated with (e.g., chemically and/or physically bound to) an isolated allele sequence of a particular genomic locus. In this manner, the signature of a dye may be linked to a genomic locus in an electropherogram. In certain embodiments, a dye is a fluorophore. Other examples include energy transfer complexes, and quenching complexes.

The term “run” refers to a single sample subjected to electrophoresis in a single capillary at one time. The same sample may be rerun later in the same or different apparatus, or at the same time in the same apparatus but using a different capillary. Thus, a run is unique to an apparatus/capillary and a particular time. Multiple runs can be conducted simultaneously, using the same detection apparatus but using different capillaries.

The term “electropherogram” refers to a graph depicting intensity over time, or of position during an electrophoresis run (e.g., on a capillary), of light emitted by a dye in an electrophoresis run. In some cases, an electropherogram presents a composite of light emitted for multiple dyes, all on the same time axis.

The light emitted by a dye is characterized by the “color spectrum” of the dye, which is the relative intensity across wavelengths of light emitted by the dye when excited.

The term “raw electropherogram data” refers to light intensities at each of a plurality of different wavelengths collected as a function of time or position in an electrophoresis run. In certain embodiments, light intensities can be measured at each of about 100 different wavelengths by a spectrophotometer. These light intensities can result from a single dye or a combination of dyes. For example if two dyes emit light at time x (or position x) and at wavelength y, the raw electropherogram intensity value for time x and wavelength y will be based on contributions from both dyes.

The intensity of light emitted by a dye can be measured as a function of the contribution to raw electropherogram data by the color spectrum of the dye. This contribution may be determined using a deconvolution process such as a matrix operation described herein. In some embodiments, the intensity of a particular dye at a particular time point in an electropherogram is represented by a scalar, which corresponds to the amount of concentration of a detectable analyte (e.g., an amplified STR) at the time point. The deconvolution may provide an absolute amount of each dye in raw data at a time point, which amount may be represented as the height of the due peak in the electropherogram. In one example, the intensity of the dye at a time point is determined by least squares fitting of the color spectrum of the dye to the electropherogram. E.g., the area under an electropherogram peak relates to the “intensity” of the dye of the “amount” of the analyte providing the dye signal.

### System Overview

In certain embodiments, the apparatus is configured for obtaining and analyzing electropherograms; the apparatus is also referred to herein as an instrument or a system. It runs and reads electropherograms. In certain embodiments, its components include capillaries, reagents, fluidics for delivering reagents to capillaries, an optical system for reading signals from dyes, and a control system for coordinating the operation of all the other components. The capillaries each include an interrogation region where fluorescent signal is generated and read for the amplicons moving through the capillary (by electrophoresis). The optical system may include an excitation source for directing excitation light to fluorophores (or other dyes that respond to light excitation) in the interrogation region of a capillary, a detection system for reading radiation emitted from fluorophores or other dyes in the interrogation region, and geometric opticals elements (e.g., lenses, mirrors, beam splitters, apertures, and the like) for coupling light from the excitation source to the interrogation region and for coupling light from the interrogation region to the detection system. The detection system may be a spectrophotometer or any other system that can detect and radiation magnitude information (e.g., radiation intensity) at multiple different wavelengths. Spectrometers are equipped with optical detectors such as a CCD or photomultiplier array. Light is separate into spectral components by a grating or similar element. An alternative detection system comprises a series of beam splitters and photodetectors, wherein the beam splitters filter light according to wavelength. Another example of a suitable detection apparatus is described in US Patent Application Publication 2016/0116439, filed Oct. 21, 2015, which is incorporated herein by reference in its entirety.

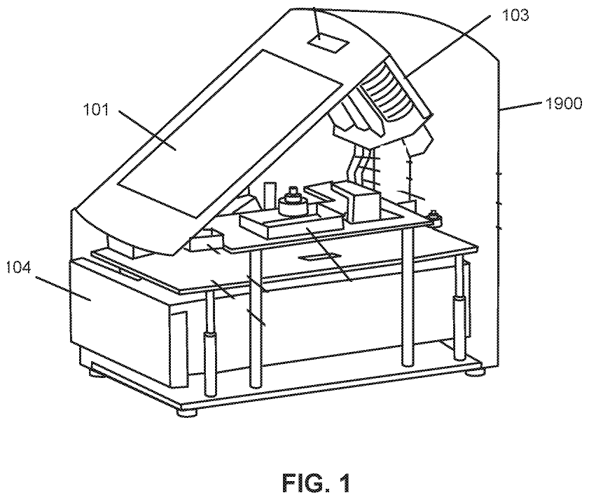

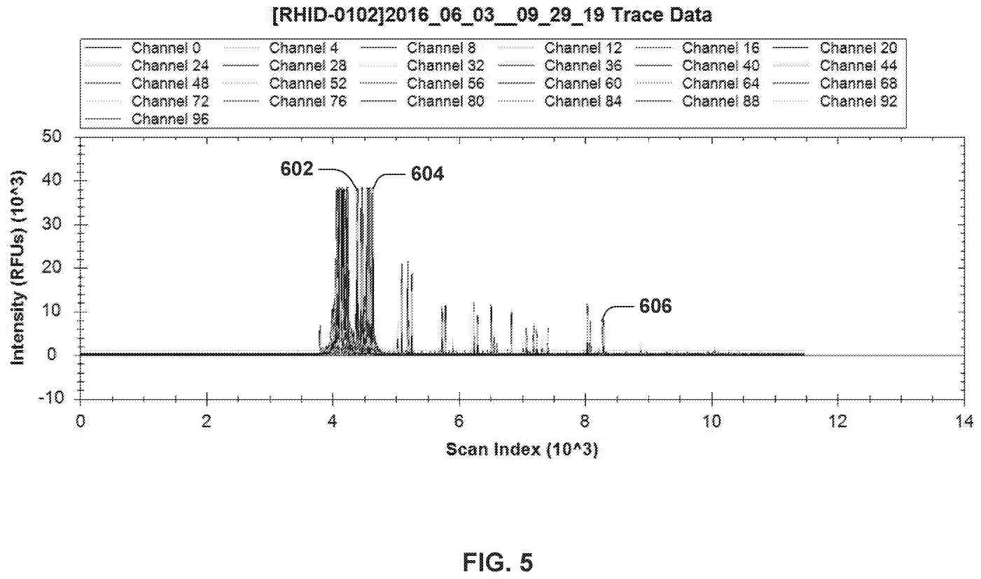

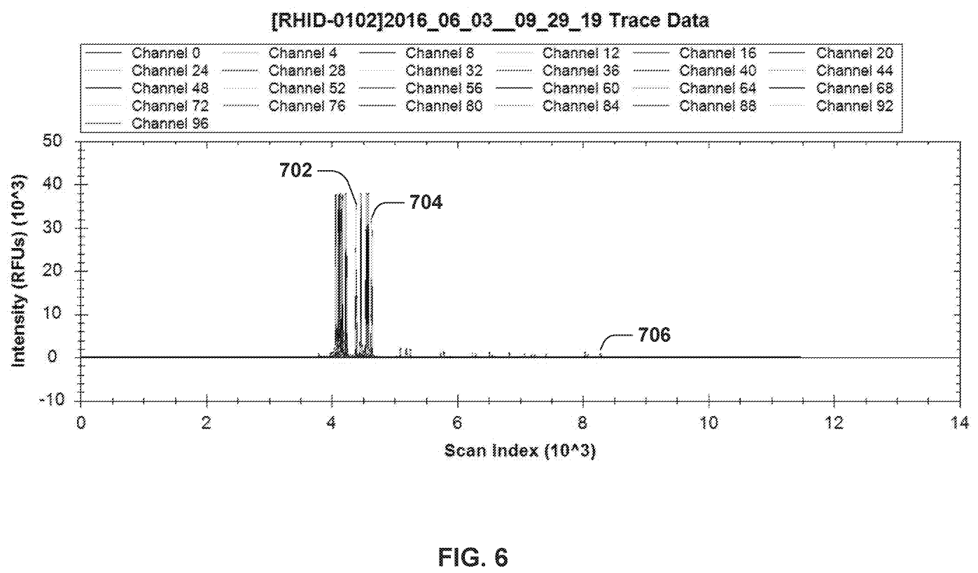

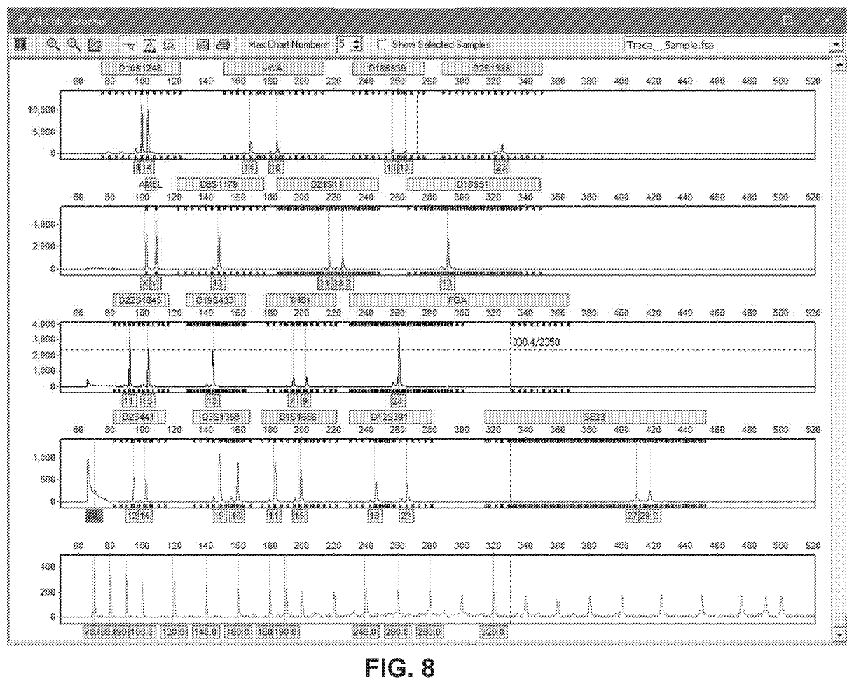

FIG. 1 shows a system for sample processing and analysis in some implementations. System 1900 can obtain electropherograms and analyze nucleic acid profiles from the electropherograms. FIGS. 5 and 6 show two examples of raw electropherogram data that can be collected. FIG. 8 shows example plot of the nucleic acid profile (electropherogram) generated from the data collected.

System 1900 can include a sample preparation sub-system, a sample analysis sub-system and a control sub-system.

A sample preparation sub-system of the system 1900 can include a sample cartridge interface 103 configured to engage a sample cartridge through slot, sources of reagents for performing a biochemical protocol, a fluidics assembly configured to move reagents within the sample preparation sub-system. A fluidics assembly can include a pump, such as a syringe pump. The pump is fluidically connectable through valves to the outlets for reagents such as water and lysis buffer and to a source of air. The pump can be configured to deliver lysis buffer and water through fluidic lines to the sample cartridge.

A sample analysis sub-system can include an electrophoresis assembly including an anode, a cathode and an electrophoresis capillary in electric and fluidic communication with the anode and cathode, and a sample inlet communicating between a sample outlet in the sample cartridge and an inlet to the capillary. These can be contained, e.g., within an electrophoresis cartridge 104. The sample analysis sub-system can further include an optical assembly including a source of coherent light, such as a laser, an optical train, including, e.g., lenses and a detector, configured to be aligned with the electrophoresis capillary and to detect an optical signal, e.g., fluorescence, therein. In an example, the electrophoresis cartridge also includes a source of electrophoresis separation medium and, in some cases sources of liquid reagents, such as water and lysis buffer, delivered through outlets in the electrophoresis cartridge to the system. Separation channels for electrophoresis can take two main forms. One form is a “capillary”, which refers to a long and typically cylindrical structure. Another is “microchannel”, which refers to a microfluidic channel in a microfluidic device, such as a microfluidic chip or plate.

A control sub-system can include a computer programmed to operate the system. The control sub-system can include user interface 101 that receives instructions from a user which are transmitted to the computer and displays information from the computer to the user. The user interface 101 may be as described in U.S. Patent Application Publication No. 2016/00116439, published Apr. 28, 2016, which is incorporated herein by reference in its entirety. In some cases, the control sub-system includes a communication system configured to send information to a remote server and to receive information from a remote server.

### Electropherogram Analysis

Methods and systems for analyzing electropherograms are further described below. While much of the discussion herein uses electropherophoresis, and particularly STR electrophoresis, as an example, the disclosed methods and systems have other applications. Generally, they apply to any biochemical or biological analytical technique that employs different dyes to identify different macromolecule loci or other biological features that are separated spatially and/or temporally. Nucleic acid sequencing and flow cytometry are examples of other analytical techniques that use different dyes in association with different biological features (e.g., unique nucleic acid sequences and unique cells) and, as such, can use the methods and systems described herein. For example, the methods and systems may help identify individual reads of a sequencer and/or different cells in a cytometer.

1. Electropherogram design: multiple loci of the genome are amplified and each locus is identified by a different fluorescent dye.

When the electrophoresis system employs more loci than dyes, some dyes are used repeatedly, and in some cases all dyes are used repeatedly. In one example, there are twenty-four loci and six dyes. In some implementations, only a single electropherogram is used. The PCR primers for each locus are attached to a dye. In this manner, particular loci are associated with particular dyes in that the PCR product (amplicon) from a genomic locus is tagged with a single dye.

Example ranges for STR electropherogram variables:

Number of dyes: one to about ten or about three to eight. For purposes of the discussion, six will often be used as an example.

Number of channels (optical wavelengths detected and binned electronically): about ten to three thousand, or about fifty to five hundred. For purposes of this discussion, 100 will often be used as an example.

Number of capillaries (lanes): about one to five hundred, e.g., about ten; some electropherogram generating apparatus available from IntegenX uses only one capillary and some use eight. When eight are used, typically seven of them are used for different samples (sometimes from seven different individuals) and one is used for a control, e.g., an allelic ladder.

Number of loci: two to about fifty, or about sixteen to twenty-six. Many more may be considered in certain nucleic acid sequencing applications.

Combinations of variables: In certain embodiments, the electrophoresis employs a number of unique loci and a number of unique dyes in a ratio of greater than 1:1. For example, the ratio may be at least about 2:1, or at least about 4:1, or at least about 8:1, and in some cases even greater than about 20:1. In certain embodiments, the electrophoresis employs a number of color channels and a number unique dyes in a ratio of at least about 1.5:1, or at least about 10:1, or at least about 15:1, or at least about 20:1.

For purposes of this discussion, twenty-four will often be used as an example. Note that a unique PCR primer pair is provided for each locus.

For sake of convenience, this disclosure will frequently refer to embodiments in which there are 100 channels, six dyes, twenty-four loci, and one capillary (lane). Any time one of these values is used, it is to be understood that other values can be substituted, particularly values within the ranges recited herein.

2. During an electrophoretic run, a spectrophotometer reads light intensity signals from an interrogation region and generates optical data in many channels (e.g., 100 channels of spectral data).

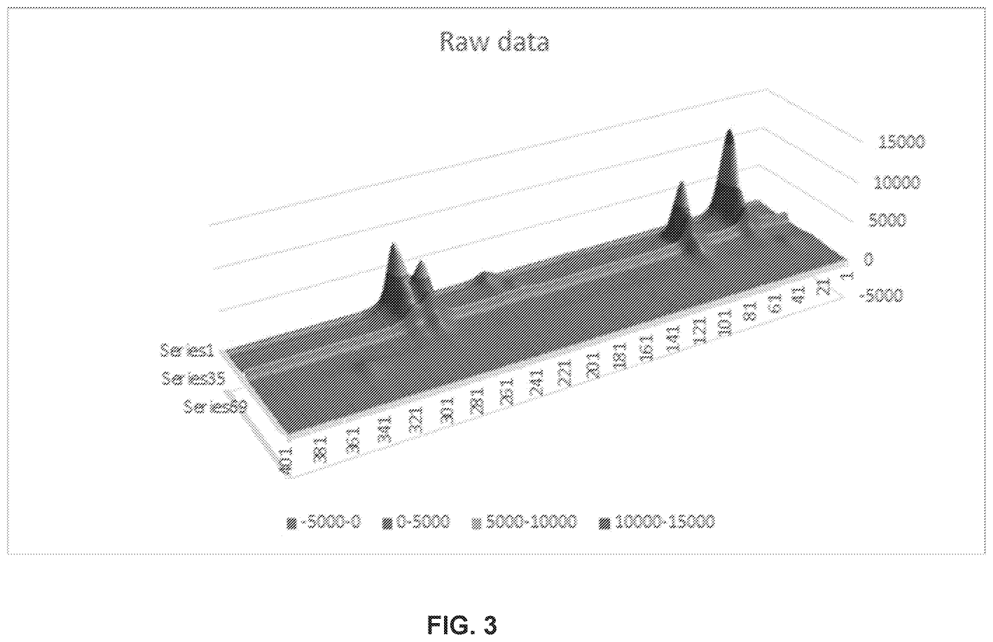

A full multi-channel data acquisition for a run contains continuous spectral emission data over many points in time at the interrogation region of an electrophoretic capillary. The resulting data is multichannel (color) magnitude values as a function of time. Time corresponds to the size (length or mass with respect charge) of the amplicon of the PCR amplified loci (e.g., STR loci). Depending on the apparatus design, there may be multiple capillary electropherograms read concurrently (e.g., about eight). Technically, the data collected during the multi-channel data acquisition may be termed raw electropherogram data. Such data contains signal intensity values as a function of wavelength and time (or position) in a capillary or other electrophoresis medium. See FIG. 3 (intensity in the z-direction and wavelength and time in the horizontal directions). Processes described herein convert the raw electropherogram data into an electropherogram, which presents intensities of individual dies as a function of time or position. In other words, the processes convert the raw intensity/wavelength data into data representing the presence of individual dyes associated with individual macromolecules separated by electrophoresis.

3. The spectrally scanned raw electropherogram data is deconvolved into different spectral peaks, each unique to a particular one of the dyes used in the process.

The raw signal provides the magnitudes of all 100 channels of the spectrophotometer (or some other number of channels depending on the spectrophotometer design) and because peaks from different dyes overlap in spectral composition and in time (which corresponds to the size and charge of the DNA amplicon fragment), multiple dyes may contribute to the signal at any instant in time. In other words, at a particular time in the raw electropherogram data, multiple dyes can contribute to the magnitude values of particular channels. To deconvolve this raw magnitude data into individual spectral peaks for the unique dyes, the process needs calibration information (e.g., a pure spectrum) for each of the dyes used in an electropherogram run.

Various deconvolution techniques are known to those of skill in the art. See, for example, J. M. Butler, Advanced Topics in Forensic DNA Typing; Methodology, pages 150-158 (2012), Elsevier, Inc., which is incorporated herein by reference in its entirety.

Calibration is used for a single instrument; i.e., the process described here is used for only a single instrument. Each instrument is separately calibrated in the manner described here. Due to changes in ambient operating conditions, such as temperature, mechanical changes create positional changes of components in the optical detection apparatus. Often these changes are large enough to require new calibration. Calibration should be conducted as often as possible, ideally once for each run.

4. Obtaining Spectra for Dyes from the Samples

The calibration information for each of the dyes used in the electropherogram is obtained from the actual samples that serve as the data for the electropherogram. This has the benefit of providing calibration that is accurate for the actual sample at hand. Compare the case where the calibration data is taken under particular conditions and at a time or under operating conditions that might not provide an appropriate representation of the calibration for the electropherogram where the calibration information is used.

In certain embodiments herein, calibration is performed separately for each run and uses exclusively calibration information (e.g., pure spectra of the dyes) from that run. In some embodiments, calibration for a run uses some information from the current run and other information from a related run. A related run may be a recent run on the same instrument, performed shortly (e.g., immediately) before or after the run under consideration. More generally, a related run may be the most recent run for which valid dye calibration data is obtained. A related run may also be a run performed at the same time and on the same instrument, but for a different electrophoresis capillary. Note that two capillaries run at the same time and with the same reagents in a single instrument may have slightly spectral shifted pure dye spectra due to geometrical differences between the two capillaries with respect to the optical system and/or other features of the instrument.

In some implementations, at least one pure spectrum is obtained for a dye, which is then used in a calibration matrix for spectral deconvolution. In some implementations, a plurality of pure spectra are obtained for a dye, which may be normalized, averaged, or otherwise combined to provide values to form the calibration matrix.

In some implementations, a pure spectrum for a particular dye may not be available from the data within a run. Under such circumstance, a pure spectrum for the particular dye may be derived from the spectrum or spectra of one or more other dyes. In some implementations, the relation of the pure spectrum of the particular dye and the spectra of the one or more other dyes may be available from a different run, or a different lane or capillary. The relation may also be available from prerecorded data obtained using similar dyes and hardware. Such relation may be used to extrapolate from the spectrum of the one or more other dyes in the run under consideration to obtain the spectrum of the particular dye.

5. Application of the Calibration Information

The raw electropherogram data to be deconvolved is provided in the form of, e.g., 100 channels of color data at a given time point. A peak in the electropherogram represents the presence of genomic data (and the dye associated with a biological feature). In some implementations, a peak comprises from about 3 to 50 time points. For purposes of further discussion the typical number of time points per peak is 10. The data in each time point is treated independently. The 100 channel color data for any point in a peak must be deconvolved into information on six distinct dyes (or as many dyes as are employed in the sample processing).

The calibration information is obtained from the sample, and, for each dye, the calibration data is represented as 100 magnitude (e.g., photometer intensity) values, one for each channel of the spectrophotometer. In other words, the calibration data for a dye contains 100 values, one signal magnitude of each channel.

Deconvolution is accomplished with, for example, the calibration data organized in the form of a calibration matrix. For a given time point in the data, the calibration matrix effectively converts a vector of 100 rows (one row for each of the data from 100 channels) to a vector of six rows (one row for each of the dyes). It does this by multiplication with a matrix of 100 columns and six rows.

The desired calibration matrix is obtained from a pseudo-inverse of a “bleed” matrix of 100 rows and six columns. The six columns represent the spectra of six different dyes that have been calibrated and the 100 rows represent the 100 channels for the spectrophotometer.

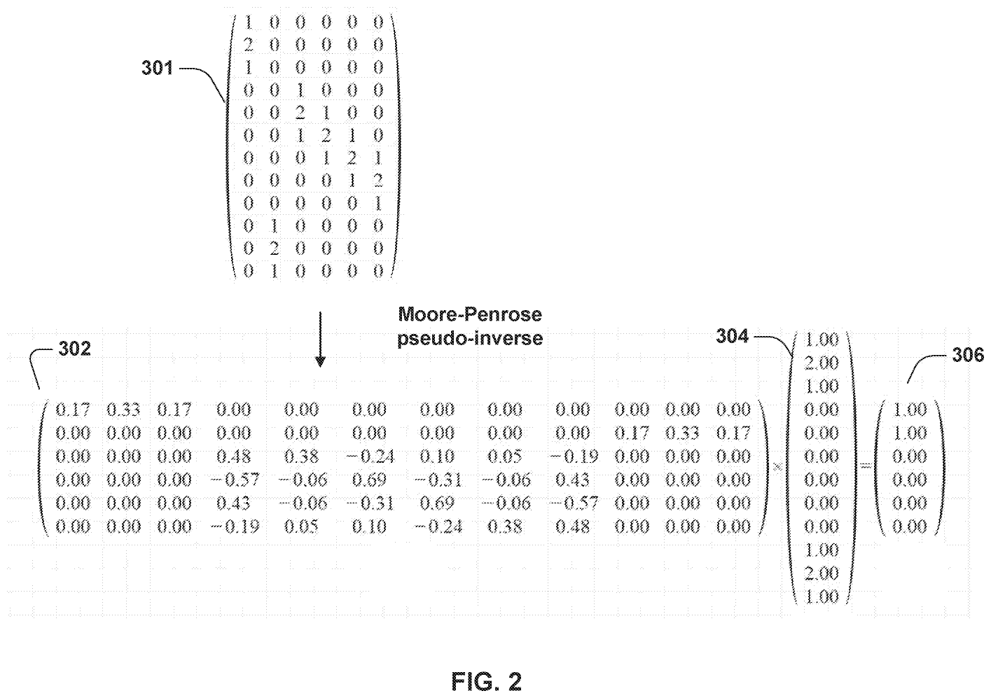

FIG. 2 schematically shows a simplified example of how a calibration matrix 302 can be obtained and used to deconvolve raw electropherogram data in a column vector 304. Matrix 301 is a “bleed” matrix having six columns, each column representing data corresponding to a pure spectrum for one of six dyes. For a simpler illustration, each column of the “bleed matrix” has only 12 rows representing 12 color channels instead of 100 rows for 100 color channels as explain in the example above. In practice, there can be 100 channels or more as described above, which can be represented by 100 rows or more in the matrix.

A first dye represented by the first column from the left in bleed matrix 301 has an intensity peak at the second color channel from the top. The color spectrum for this first dye has values 1, 2, and 1 in the first three color channels. The dye represented by the second column has a color spectrum with a peak at the 11th color channel, with signal amplitudes of 1, 2, and 1 at the 10th, 11th, and 12th color channels. A third dye represented by the third column in the calibration matrix has a peak in the fifth color channel. Similarly, a fourth dye presented by the fourth column in the calibration matrix has a color spectrum peak at the sixth color channel. The fifth dye presented by the fifth column in the calibration matrix has a color spectrum peak at the seventh color channel. A sixth dye presented by the sixth column of the calibration matrix has a color spectrum with a peak at the eighth color channel.

The column vector 304 illustrates a simplified example of a column vector representing raw electropherogram data for a single time point. The column vector 304 has 12 rows, each row presenting electropherogram data for one color channel.

The raw data represented by the column vector 304 includes a peak centered on the second color channel (starting from the top), having schematic data values of 1, 2, and 1 in the first three color channels. In practice, the real data values can be different, such as values up to many thousands in RFU. The raw electropherogram data represented by column vector 304 also includes a peak at channel at the 11th channel, with data values 1, 2, and 1 at the 10th to the 12th color channels.

To deconvolve the column vector 304 of raw data to obtain values for each dye, a calibration matrix 302 can be obtained from bleed matrix 301 by a Moore-Penrose pseudo-inverse in some implementations. In other implementations, a single value decomposition technique may be used to obtain the calibration matrix 302 from the bleed matrix 301.

The calibration matrix 302 has six rows, each row for a dye. The calibration matrix 302 has 12 columns, each column for a color channel. When the calibration matrix 302 is multiplied by the column vector 304 of the raw electropherogram data, the column vector 306 having six rows is obtained, each row resenting the intensity or amplitude of the signal detected for one of the six dyes. The values of the column vector may be normalized for downstream processing. In this simplified example, it can be seen that the column vector 304 has a peak at the second color channel from the top and the 11th color channel from the bottom from the top. These two peaks correspond to the spectral peaks of the two dyes presented by the first and second rows of calibration matrix. As such, the column vector 306 provides values of the six dyes after deconvoluting the raw data of the column vector 304.

6. How the Calibration Data for Each Dye is Obtained

In this disclosure, the calibration data is a spectrum for each dye. Finding such spectra relies on finding spectral peaks associated with a single dye, uncontaminated by other dyes. The calibration data used to create the calibration matrix is in the form of a dye spectrum for each dye used in a run. Each dye spectrum contains magnitude values for each of the color channels (wavelengths). Frequently, the non-zero values are concentrated in a relatively small spectral region.

In certain embodiments, the dye spectra are obtained from the sample raw electropherogram data by identifying particular color peaks (e.g., intensity as a function of wavelength at a particular time point) that are determined to be uncontaminated by signal from other dyes. Uncontaminated color peaks are identified by considering signal information in the form of raw data peaks in three dimensions, with one dimension being the magnitude (typically signal intensity) of the peak (or the magnitudes of the readings in the color channels that make up the peak), another dimension being the time when the peak was recorded (e.g., at the interrogation region of a capillary), and the third dimension being the wavelength/color information associated with the peak. Peaks can be resolved in time by simply identifying groups of high magnitude values that collectively have a significant slope and are reasonably separated (in time) from the nearest other magnitude peaks.

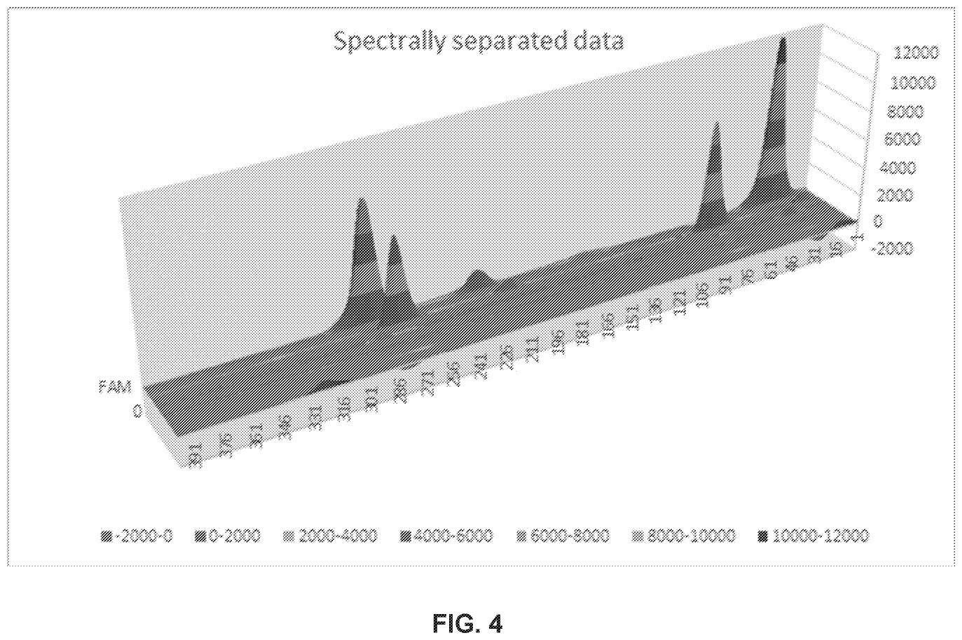

The raw electropherogram data may be provided in a three dimensional array. Each data point comprises a time value with the intensity of 100 binned colors recorded from the spectrometer. See FIG. 3, where the vertical (z-direction) axis represents signal intensity (magnitude), the long horizontal axis represents time (or position), and the short horizontal axis represents color or wavelength. A trace of such data points is converted into a three dimensional array replacing 100 colors with 6 dye intensities. See FIG. 4, where different color bands represent different dyes. This result may be considered to be an electropherogram.

To identify potential pure dye peaks, the magnitude data can be characterized based on slope in the wavelength dimension. The wavelength dimension is divided into positions based on the color channels of the spectrophotometer. In the example shown in FIG. 3, 100 channels are used. The channels are ordered sequentially by wavelength.

Because dyes emit radiation in a narrow band of wavelengths, color peaks that have steep slopes in the wavelength dimension suggest that the colors in the peak are from a single dye. Such peaks are candidates for calibration of the dye they represent. Various criteria in the wavelength dimension may be considered. For example, the rising and/or falling edges of the peak may be required to increase and decrease monotonically. Additionally, the rising and/or falling edge(s) may need to have a slope of at least a predefined value.

Further, the peaks may be selected to have means or other central tendencies at wavelengths known to be emitted by particular dyes. If the characteristic wavelength of a color peak is more than a threshold distance (e.g., 5 or 10 nm) from the wavelength of a dye under consideration, then the color peak is discarded from consideration.

For each dye, the process identifies one or more potentially spectrally-pure color peaks. Again, it does this by identifying color peaks that are spectrally and temporally compact and reasonably separated in time from nearest neighbor peaks. In some cases where multiple candidate color peaks are identified, a limited number are identified for use in calibration. In one embodiment, the process selects peaks of a particular dye from the candidates by considering the correlation between the spectra of the candidate peaks. Those color peaks showing the strongest correlation are selected for use in calibration. For example, the ten most correlated color peaks are used, or the five most correlated color peaks are used, or the three most correlated color peaks are used, or the two most correlated color peaks are used. In alternative embodiments, only a single color peak is used. In some cases, the process will consider as many color peaks as meet the compactness and separation criteria (or whatever criteria are used to select candidates). If only a single color peak is identified, then that peak alone will be used for the calibration of the dye under consideration.

Various statistical measurements of correlation may be used to correlate two spectra, including but not limited to Pearson's correlation coefficient, Spearman's rank correlation coefficient, Kendall tau rank correlation coefficient, randomized dependence coefficient, polychoric correlation and other distance correlation techniques.

If multiple candidate color peaks are selected for use in the dye spectrum for calibration, the spectra of the selected peaks may be averaged or otherwise combined to provide a single dye spectrum. In one example, the spectra of each selected color peak are normalized and then averaged on a channel-by-channel basis. In other words, the magnitude values for each channel of the selected color peaks are averaged to provide an averaged dye spectrum for use in calibration. In some cases the peaks are averaged using a weighted average, where the weights may be determined by a parameter associated with likely reliability of the color peaks. Examples of such parameters include (i) magnitude (e.g., signal intensity) of the centroid, mean, or other central tendency of the color peak, with larger magnitudes being given greater weight, and (ii) peak width, with narrower peaks being given greater weight, and the like. In some examples, three separate peaks for a dye are combined to prepare a pure spectrum for the dye. The final dye spectra are used in a calibration matrix as described above.

7. Backup Analysis

When individual dye peaks cannot be identified from a sample, a backup procedure may be employed. This situation may arise in very complex samples such as “allelic ladders,” which are test samples containing all possible alleles of a sample. Such samples have so many peaks in all dye colors, that it is very difficult to find any that are pure (i.e., that can result from a single dye).

Process Steps for Each Dye:

a. determine whether the current sample contains information to generate a pure spectrum by a method such as those described above. If so, use the pure spectrum for calibrating the current sample.

b. if (a) does not hold, use the calibration information from the most recent sample taken with the instrument. However, scale it, if necessary, to account for possible variations between the time when the calibration was made and the current time.

Note that the calibration data need not be taken from the most recent sample analyzed with the instrument. It may be taken from a subsequently analyzed sample in the instrument. Or it may be taken from another capillary used in a concurrent or prior run. As noted, some instruments are configured to run multiple samples concurrently. Ideally, each capillary run is used to produce its own calibration information for each dye.

c. identify scaling—identify at least one different dye (i.e., one having a different color from the one or more under consideration) for which a pure spectrum can be produced from the current sample. In other words, the process identifies a dye that meets the requirements of (a). The pure spectrum of that dye from the current sample is compared to the pure spectrum of that dye from the most recent sample, the one used for calibrating the current sample. The relationship between the spectra of the dye taken in the current sample and the recent sample defines a scaling that is applied to the pure spectra of other dyes taken from the recent sample (i.e., of dyes that do not meet the requirements of (a)). The scaled versions of these prior determined dye spectra are used in calibrating the current sample. Scaling may involve a spectral shift and/or a change in the shape of a peak.

Note that in some embodiments, every run includes a calibrant containing DNA fragments of known length and having known dyes. Some of these fragments are larger than those of any alleles that could be found in a sample. As a consequence, the color peaks found in the region of the data associated with such large fragments are guaranteed to contain signal from only a single dye (e.g., orange). The signal from such color peaks is used to identify the pure spectrum for the dye that produced the color peak. This spectrum can be used in the calibration matrix for the sample under consideration. It can also be used for scaling spectra for dyes that do not meet the requirements of (a).

Note that in other embodiments an additional spectral calibration dye may be bonded to DNA fragments and run with a sample. The lengths of such calibration fragments is substantially different from any contained in the sample. This calibration dye emits light in substantially different wavelength regions from those dyes bonded to the PCR product (or other macromolecule) from the sample. The data from this additional calibration dye may be used as above to scale spectra for dyes that do not meet requirement of (a).

d. for any pure dye spectra taken from related runs, apply any scaling (e.g., shift in wavelength or a change in the shape of the spectrum) identified for pure spectra. A shift may be an observed variation in the central tendency (e.g., mean or median) or centroid or other peak feature that is a function of wavelength. Spectral shape modifications can be made in several ways. One methodology is to normalize each dye spectra then multiply the original spectral shape by the scaled difference prior and current between the scaling dye identified in (c).

8. Signal Saturation

In some experimental conditions and hardware environments, there is a signal saturation problem in capturing and analyzing electropherogram data. On the one hand, if light is captured by the light detection sensor using a long exposure time, signals can become saturated for high-intensity data. On the other hand, if the light is captured with a short exposure time, signals may be too low in intensity or signal-to-noise ratio to provide good results. FIG. 5 and FIG. 6 show two electropherogram data plots illustrating this problem. In FIG. 5, the raw electropherogram data is captured with a long exposure time. The horizontal axis illustrates scan index indicative of sizes of DNA molecules. The vertical axis illustrates signal intensity in relative fluorescent units (RFU).

The detected data in the range between about 5 to about 9 on the horizontal axis have relatively good intensity levels. It is not the same when the same sample signals are captured using a shorter exposure time as shown in FIG. 6. The data peak 606 in FIG. 5 corresponds to the data peak 706 in FIG. 6. The data peak 606 has relatively strong intensity that can provide good signal for an electropherogram analysis. In contrast, the data peak 706 in FIG. 6 is low, which may be inadequate to provide sufficient signal.

The long exposure data shown in FIG. 5 provides good signals in the range between about 5 to about 9 on the horizontal axis. However, the data in the range between about 4 to about 5 of the horizontal axis have an opposite problem. Signal peaks 602 and 604 in FIG. 5 are saturated. This saturation causes information loss, making it impossible to distinguish the difference in signal strengths between data peaks 602 and 604. The electropherogram data peaks 702 and 704 in FIG. 6 respectively correspond to peaks 602 and 604 in FIG. 5. Data peaks 702 and 704 have good signal strength and are not saturated, and a difference between the two peaks is clearly visible.

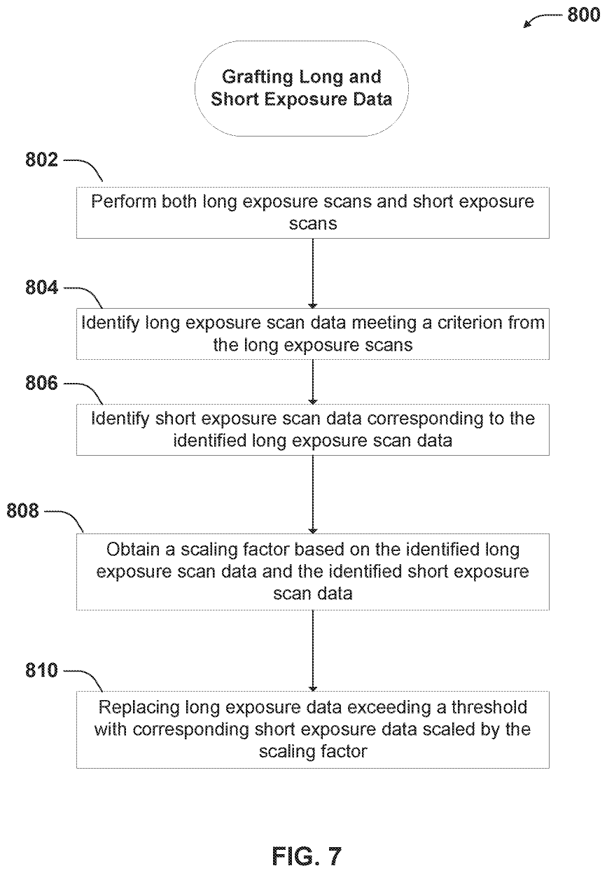

Some implementations solve this problem by grafting long exposure scan data with short exposure scan data, thereby extending the effective dynamic range of the data captured by the system. FIG. 7 illustrates a process 800 for grafting long exposure and short exposure scan data according to some implementations. Process 800 utilizes short exposure data when the long exposure data is saturated. In various implementations, this process is performed before spectral devonvolution described above.

Process 800 starts by recording both long exposure scans and short exposure scans. See block 802. In some implementations, the short exposure time is about 10 ms and the long exposure time is about 100 ms. Other values of short exposure time and long exposure time may be used depending on the operating characteristics of the hardware and data processing pipeline.

Process 800 proceeds to identify long exposure scan data meeting a criterion from the long exposure scans recorded in operation 802. See block 804. In some implementations, the signal level of the long exposure data meets a signal level threshold or falls in a signal level range. In some implementations, the long exposure scan data have a signal level between 10000 to 25000 RFUs. In some implementations, the signal level of the wavelength channel 25, or the channel corresponding to a specific PCR primer dye, is used with reference to the criterion range or the criterion level. In some implementations, the signal level of channels other than channel 25 is used. In some implementations, the scan data is identified when a Raman line is present and a laser is turned on. In some implementations, signal levels in different ranges or at different levels may be used to identify the data. For instance, in some implementations, the signal level is between 5000 to 30,000 RFUs. In some implementations, the signal level is between 4000 and 35,000 RFUs. In some implementations, the values of the signal levels are chosen to ensure that the signal of long exposure time is relatively large but not saturated, while at the same time is not too small so that a corresponding scan of a short exposure time still has a sufficient signal level.

In some implementations, a plurality of scans is obtained and the data from the plurality of scans are averaged to obtain a scaling factor as further described below. In some implementations, 100 scans are identified. In some implementations, 10, 20, 30, 40, 50, 100, 200, 500, 1000, and 5000, scans are identified. In some implementations, when not enough scans as stated above can be identified, a smaller number of scans may be used.

Process 800 further involves identifying short exposure scan data corresponding to the identified long exposure scan data. See block 806. In some implementations, the short exposure scan data are identified based on a temporal proximity or relation with the long exposure scan data. For example, short exposure time raw data may be aligned in time with the long exposure time raw data using linear interpolation. In some implementations, short exposure data may be associated with long exposure data by various correlation techniques described elsewhere herein.

Process 800 proceeds to obtain a scaling factor based on the identified long exposure scan data and the identified short exposure scan data. See block 808. In some implementations, the scaling factor is a ratio between the long exposure data and the corresponding short exposure data. In some implementations, the scaling factor is a difference between the two data. In some implementations, the scaling factor is selected from other quantities reflecting the relation between the long exposure data and the short exposure scan data. In some implementations, the scaling factor may be a function relating the short exposure scan data to the long exposure scan data. In some implementations, a plurality of the long exposure scan data and a plurality of the short exposure scan data are used to obtain a plurality of ratios, and an average value of the plurality of ratios is used as the scaling factor. In some implementations, the plurality of the long exposure scan data and the plurality of the short exposure scan data are used to obtain a relation or a function between the two data, and the relation or the function is used as the scaling factor.

Process 800 then proceeds to replace long exposure data recorded in 802 with corresponding short exposure data scaled by the scaling factor, the replaced data have signal levels exceeding a threshold value. In some implementation where the scaling factor is a ratio, the scaled data is obtained by multiplying the short exposure data by the scaling factor. In some implementations where the scaling factor is a function, the scaling data is obtained by applying the function to the short exposure scan data. In effect, process 800 grafts the long exposure data and the short exposure data to achieve a larger dynamic range.

The grafted electropherogram data may then be further analyzed to obtain nucleic acid profiles. FIG. 8 shows an example of nucleic acid profiles obtained using such grafted data electropherogram data.

### EXAMPLES

### Example Processing Pipeline

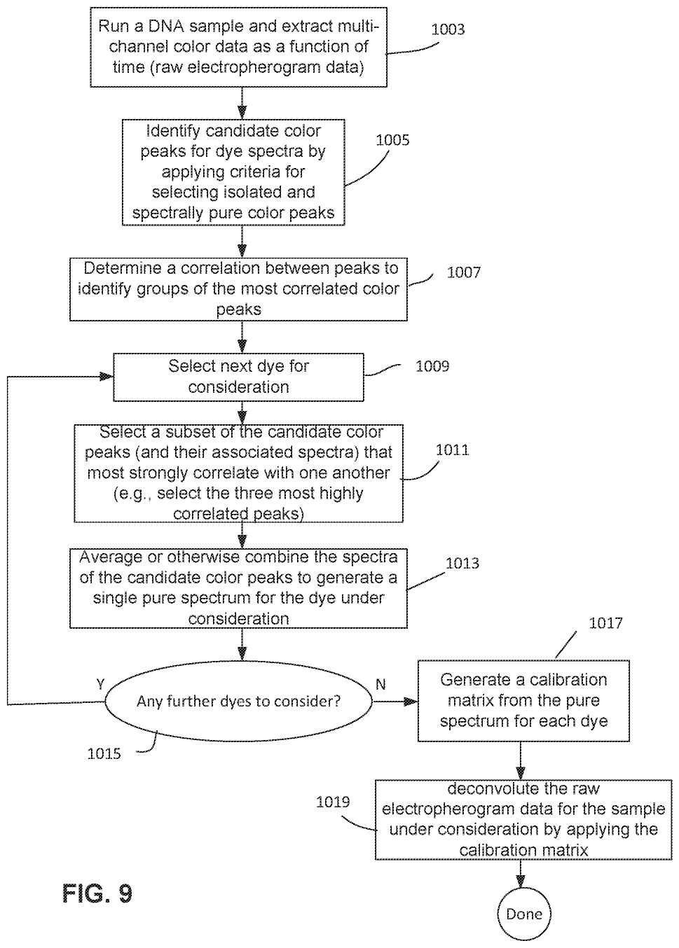

An example processing pipeline for spectral deconvolution is depicted in FIG. 9. In this example, one or more dyes are calibrated using the current sample's electropherogram.

1. Run a sample and extract multi-channel color data as a function of time (an electropherogram). See operation 1003. In certain embodiments, all color data from all channels is used. See, e.g., FIG. 3.

2. Identify candidate color peaks for dye spectra by applying criteria for selecting isolated and spectrally pure peaks. See operation 1005. In certain embodiments, this is accomplished by identifying intensity peaks in the multi-channel raw electrophoresis data. Candidate color peaks may be required to have a specified threshold intensity level, which may be selected empirically. In one example, the threshold is chosen to remove candidate color peaks that are likely noise.

Candidate color peaks may be required to increase or decrease monotonically in the wavelength dimension. In other words, at a point in time, a candidate color peak should have monotonically increasing values of intensity as the wavelength increases toward the peak or monotonically decreasing values of intensity as the wavelength decreases away from the peak. The monotonicity requirement may apply to one or both sides of a color peak and may apply for a certain distance from the peak. For example, a monotonicity check may require a monotonic decrease from a peak's maximum down to 15% of the peak height on both sides of the peak.

Still further, a candidate color peak should be centered at or near a wavelength known to be emitted by one of the dyes for which pure spectra are sought. For example, if the wavelength of a candidate color peak is not within about 5 nm of the wavelength of any of the expected maximum intensities of the dyes under consideration, the candidate color peak may be discarded from further consideration.

3. Determine a correlation between peaks to identify groups of the most correlated peaks, each group possibly representing spectrally pure peaks for a single dye. See operation 1007.

In some embodiments, the color peaks are first segregated into those for particular dyes based on wavelength and after this segregation the correlation is applied. Alternatively, all candidate color peaks are analyzed by cross-correlation and this process itself self-segregates the peaks associated with particular dyes.

4. For each dye (operation 1009)

a. select a subset of the candidate color peaks (and their associated spectra) that most strongly correlate with one another (e.g., select the three most highly correlated peaks). See operation 1011.

b. average or otherwise combine the spectra of the candidate color peaks to generate a single pure spectrum for the dye under consideration. See operation 1013.

5. Generate a calibration matrix from the pure spectra for each. See operation 1017.

6. deconvolve the raw electropherogram data for the sample under consideration by applying the calibration matrix to the raw electropherogram data. See operation 1019.

In some implementations, operations 1005, 1007, 1011, and 1013 are performed sequentially for a single dye. In other words, operations 1005 and 1007 are performed for a single dye (they identify candidate color peaks for only one dye at a time). When operation 1013 is complete (a pure spectrum for one dye is obtained), the process loops back to operation 1005 where candidate color peaks are identified for the next dye under consideration.

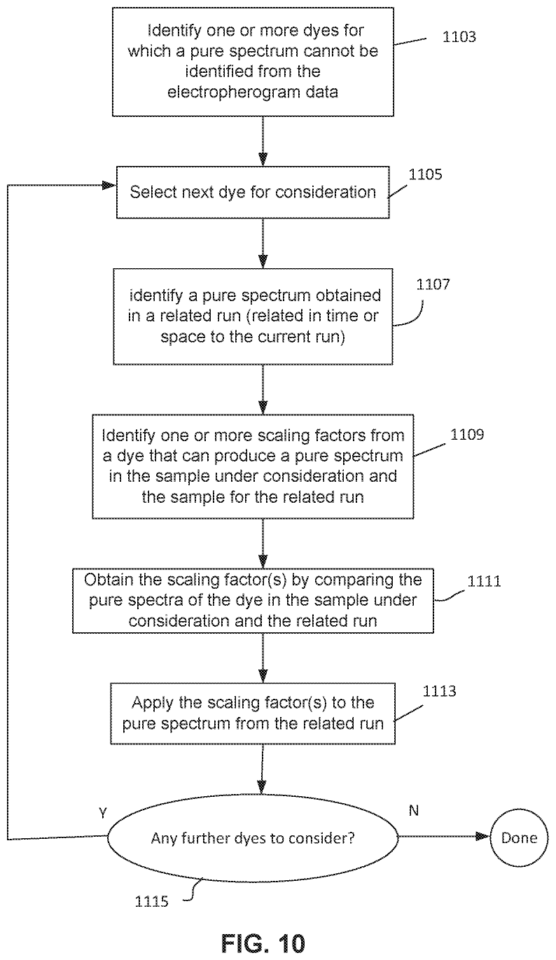

Another example processing pipeline is depicted in FIG. 10. In this example, at least one dye is calibrated using the current sample's electropherogram, and at least one other dye is calibrated using a related sample's electropherogram.

1. Identify one or more dyes for which a pure spectrum cannot be identified from the electropherogram data. See operation 1103.

For each such dye,

2. identify a pure spectrum obtained in a related run (related in time or space to the current run). See operation 1107.

3. optionally scale the pure spectra identified in 2.

Identify one or more scaling factors from a dye that can produce a pure spectrum in the sample under consideration and the sample for the related run. See operation 1109. Obtain the scaling factor(s) by comparing the pure spectra of the dye in the sample under consideration and the related run. See operation 1111.

Apply the scaling factor(s) to the pure spectrum from the related run. See operation 1113.

### Example System

In some implementations a system comprises several integrated modules, including an analyte preparation module, a detection and analysis module and a control module.

### Analyte Preparation Module

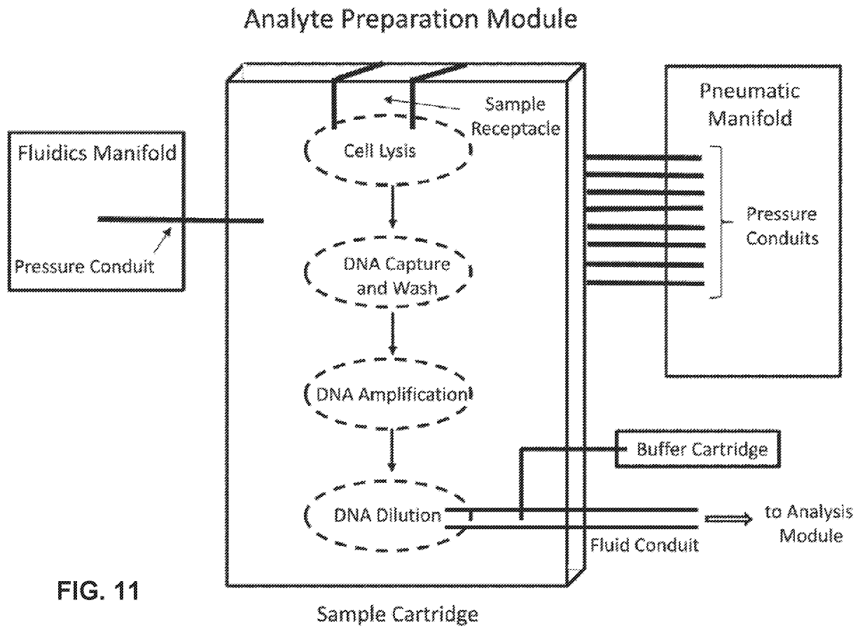

FIG. 11 shows an embodiment of an analyte preparation module of some implementations. An analyte preparation module can comprise a sample cartridge module that receives a sample cartridge and is configured to move fluids within the cartridge. The sample cartridge comprises a sample receptacle to receive a sample and areas to perform functions such as cell lysis, DNA capture and wash, DNA amplification and DNA dilution. A fluidics manifold connected to a source of pressure can deliver pressure, e.g., air pressure, into the cartridge to move liquids within the sample cartridge. A reagent cartridge connected to a source of pressure can move reagents, such as buffer and/or water into the sample cartridge. Sample and buffer can be moved out of the cartridge through a fluid conduit to an analysis assembly. In one embodiment, the sample cartridge comprises a fluidic chip that comprises a fluidics layer comprising fluidic channels, an actuation layer comprising actuation channels and an elastomer layer sandwiched between them. The chip can include valves and pumps actuated by the actuation layer. In such an embodiment, the sample cartridge module can include a pneumatic manifold connected to a source of pressure that transmits pressure to the cartridge pneumatics when the manifold engages the cartridge. This pneumatic pressure can operate pumps and valves in the cartridge to move fluids around the cartridge and out of the cartridge.

Further details of the elements and features of an analyte preparation module are described in U.S. Pat. No. 8,894,946, which is incorporated by reference in its entirety.

### Analysis and Detection Module

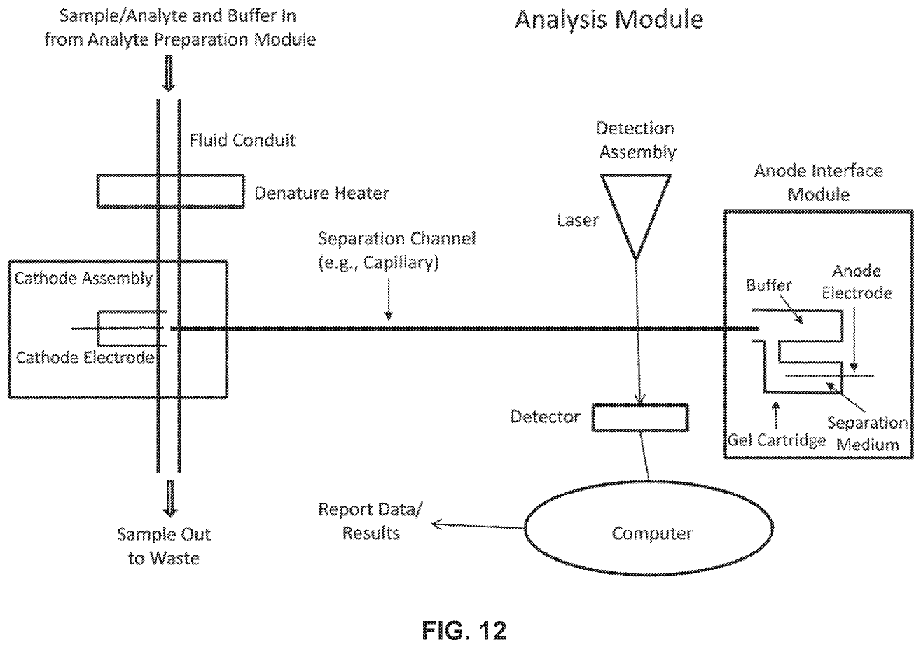

FIG. 12 shows an analysis and detection module including (1) a capillary electrophoresis assembly, (2) a detection assembly and (3) an analysis assembly.

Sample (e.g., amplified DNA or controls) and buffer (e.g., electrophoresis buffer) flow through a fluidic conduit, such as a tube, from an analyte preparation module in a path that can include a denature heater, a cathode assembly for injecting analyte into a capillary, and out to waste. A denature heater heats fluid containing DNA and denatures strands in double stranded DNA into single strands. The cathode assembly can include an electrode, such as a forked electrode, connected to a source of voltage. When a sample to be analyzed is positioned for injection, the electrode can provide voltage to inject the analyte into the capillary. The capillary is filled with a separation medium, such as linear polyacrylamide (e.g., LPA V2e, available from IntegenX Inc., Pleasanton, Calif.). The capillary ends are electrically connected to a voltage source, e.g., an anode and a cathode.

Separated analyte is detected with a detection module. The detection module can employ, for example, a laser and a detector, such as a CCD camera, CMOS, photomultiplier, or photodiode. The anode assembly (e.g., anode cartridge interface) can include an anode in electrical connection with the capillary and a source of voltage. The anode assembly also can include a source of separation medium and a source of pressure for introducing separation medium into a capillary. The anode assembly can include electrophoresis buffer. The separation medium and/or the electrophoresis buffer can be included in an anode cartridge. The anode cartridge can be configured for removable insertion into the anode assembly. It can contain separation medium and/or electrophoresis buffer sufficient for one or more than one run.

### Capillary Electrophoresis Assembly

The capillary electrophoresis assembly can include an injection assembly that can include a denature assembly, a cathode assembly; a capillary assembly; an anode assembly; a capillary filling assembly for filling a capillary with separation medium; a positioning assembly for positioning an analyte (or sample) for capillary injection; and a power source for applying a voltage between the anode and the cathode.

The capillary electrophoresis system can include one or more capillaries for facilitating sample or product separation, which can aid in analysis. In some embodiments, a fluid flow path directs a sample or product from the cartridge to an intersection between the fluid flow path and a separation channel. The sample is directed from the fluid flow path to the separation channel, and is directed through the separation channel with the aid of an electric field, as can be generated upon the application of an electrical potential across an anode and a cathode of the system. U.S. Pat. No. 8,894,946 provides examples of electrophoresis capillaries for use in analysis, as may be used with systems herein. The capillary can be inserted into the fluidic conduit for fluidic and electric communication.

### Detection Assembly

A detector can be used to observe or monitor materials in the electrophoresis capillaries (or channels). The detector can be, e.g., a charge-coupled device (CCD) camera-based system or a complementary metal oxide semiconductor (CMOS) camera-based system.

In some implementations, the system includes a single electrophoresis channel or capillary. U.S. Patent Application Publication No. 2016/0116439 describes such a system, which is incorporation by reference in its entirety for all purposes.

In other implementations, the system includes multiple (e.g., 4, 8, 10, 16, 24, 32, 40, 48 or more) electrophoresis separation channels (e.g., capillaries). U.S. Pat. No. 8,894,946 describes such a system. The system also includes a light source (e.g., a laser device or a light-emitting diode), an optical detector, and an optical selector. The laser device is positioned to deliver a beam from the laser device to at least one electrophoresis capillary. The optical detector is optically coupled to receive an optical signal from at least one electrophoresis capillary. The laser device, optical detector, and optical selector are in an arrangement that allows the optical detector to selectively detect an optical signal from any one or more of the multiple electrophoresis capillaries.

The laser device can be selected in part based on an output wavelength suitable for distinguishing the separated analyte (e.g., nucleic acid fragments). The nucleic acid fragments can be labeled with a certain number of (e.g., 2, 3, 4, 5 or more) spectrally resolvable fluorescent dyes (e.g., by using PCR primers labeled with those dyes in amplification) so that fragments having different sequences but having the same size and the same electrophoretic mobility can still be distinguished from one another by virtue of being labeled with dyes having spectrally resolvable emission spectra. The laser device can be selected to have one or two output wavelengths that efficiently excite the fluorescent dyes used to label the nucleic acid fragments. The laser device can have a single output wavelength (e.g., about 488 nm) or dual wavelengths (e.g., about 488 nm and about 514 nm). The laser device can scan across the interior of each separation channel at an appropriate rate (e.g., about 1 Hz to about 5 Hz, or about 2 or 3 Hz). The fluorescence emission of each dye excited by the laser device can pass through a filter and a prism and can be imaged onto, e.g., a CCD camera or a CMOS camera.

In one embodiment, the capillaries are arranged as an array. In one embodiment, the optical selector is optically positioned between the laser device and the multiple electrophoresis capillaries. The beam from the laser device is delivered to a single electrophoresis capillary and not delivered to other electrophoresis capillaries. In one embodiment, the optical selector is a scanning objective directing the beam from the laser device to the single electrophoresis capillary and not to other electrophoresis capillaries. In one embodiment, the scanning objective is adapted to make a traversing motion relative to the beam from the laser device entering the scanning objective. In another embodiment, the optical selector is an aperture passing the beam from the laser device to the single electrophoresis capillary and not to other electrophoresis capillaries. One embodiment further includes a capillary alignment detector optically coupled to receive a reflection of the beam from the single electrophoresis capillary. The reflection indicates an alignment of the beam with the single electrophoresis capillary.

In one embodiment, the optical selector is optically positioned between one or more electrophoresis capillaries and the optical detector. The optical signal from the multiple electrophoresis capillaries to the optical detector is limited to a single electrophoresis capillary.

Various embodiments further include a wavelength dependent beam combiner optically coupled between the laser device and the optical detector, or a spatial beam combiner optically coupled between the laser device and the optical detector.

### Analysis Assembly

An analysis assembly can comprise a computer comprising memory and a processor for executing code in the computer for receiving the data output of the detection assembly, processing the data and producing a file that reports a metric or characteristic of the analyte(s) analyzed (e.g., an answer).

In one embodiment, the analysis module can comprise memory and a processor that executes code that performs the analysis to classify STR fragments by length and by the spectral characteristics of an attached dye and then use this information along with ancillary information such as the separation of an allelic ladder to determine which STR alleles are present in the detected amplification products; this process is typically referred to as calling the STR alleles. In the case of STR analysis, the analysis assembly can receive raw electropherogram data, transform it into a format that is recognizable by, e.g., allele calling software, and, using the allele calling software, identify alleles and report them in a format understandable by a user or recognized by a database. For example, the analysis assembly can take an electropherogram and produce a CODIS file recognized by, e.g., the FBI's National DNA Index System (NDIS).

An electropherogram generated from separation of amplified STR fragments can be analyzed by the system using spectral deconvolution methods as further described hereinafter. The spectral deconvolution methods deconvolve the color data of the electropherogram to separate the contributions of each of the dyes to the electropherogram.

The detection modality of the system (e.g., optical detection) will produce a data stream that is an amalgam of the signals coming from fluorescent dyes attached to the STR fragments as well as a host of optical and electronic background effects. This data stream can be processed into a form that is consumable by the STR calling software (e.g., an expert system).

The input data that is expected by most commercial STR-calling expert systems typically contains arrays of numbers of dimensionality N×M, where N is the number of dyes that are detected by the system, and M is a time sequence of points taken during the separation. Some expert systems have upper limits on N and M, and this can vary from product to product. There are a number of ancillary assumptions that commercial expert systems make about these data streams:

(1) Most electronic and optical noise from the detection mode has been removed.

(2) Each of the N channels nominally referenced to the same dark signal, defined to be “zero.”

(3) Enough measurements have been taken of each fragment to insure sufficient base-pair resolution for the minimum-size repeat pattern in the STR kit. Nominally, this means a sampling frequency sufficient to obtain 5-10 measurements over the time that it takes a fragment to migrate past the detector.

(4) Each individual channel in the N dimension represents the photonic signal coming from a single dye as much as is possible for the detection mode. To the degree that this condition isn't satisfied, it is called “bleed-through”.

The functionality that STR calling software can provide includes:

(1) Sizing of fragments relative to an in-lane size standard.

(2) Calibration of allele bins using a (potentially optional) allelic ladder.

(3) Allele calling with morphological rejection filters (for common PCR effects such as stutter).

(4) Quality flag assignment based on mathematical measures such as signal-to-noise.

(5) Call summary output generation as text.

The practitioner can to properly tune the performance of the STR calling software to minimize the false-positive measurement set. The procedures for this are known in the art and, for commercially available software, can be contained in the product documentation.

As described above, expert systems will provide services that identify the base pair size of fragments found in the data stream and attach a preliminary allele assignment to each fragment if such exists. In addition, a quality flag can be assigned to the allele call which is reported to the analyst. The practitioner then decides what the STR profile actually is based on information from the flags. The process can be further automated by putting into place a rules engine to process the calls and quality flags into a final profile. This rules engine can be trained on the system's data to know when to keep and when to reject an allele based on the specific content of the quality flags coming from the system.

### Control Module

Systems provided herein include a control module implemented using various hardware and/or software. In some embodiments, a system for sample preparation, processing and analysis includes a controller with a central processing unit, memory (random-access memory and/or read-only memory), a communications interface, a data storage unit and a display. The communications interface includes a network interface for enabling a system to interact with an intranet, including other systems and subsystems, and the Internet, including the World Wide Web. The data storage unit includes one or more hard disks and/or cache for data transfer and storage. The data storage unit may include one or more databases, such as a relational database. In some cases, the system further includes a data warehouse for storing information, such user information (e.g., profiles) and results. In some cases, the data warehouse resides on a computer system remote from the system. In some embodiments, the system may include a relational database and one or more servers, such as, for example, data servers. The system may include one or more communication ports (COM PORTS), one or more input/output (I/O) modules, such as an I/O interface. The processor may be a central processing unit (CPU) or a plurality of CPU's for parallel processing.

The system may be configured for data mining and extract, transform and load

(ETL) operations, which may permit the system to load information from a raw data source (or mined data) into a data warehouse. The data warehouse may be configured for use with a business intelligence system (e.g., Microstrategy®, Business Objects®). It also can be configured for use with a forensic database such as the National DNA Index System (NDIS)) in the USA or NDAD in the United Kingdom, State DNA Index Systems (SDIS), or Local DNA Index Systems (LDIS) or other databases that contain profiles from known and unknown subjects, forensics samples, or other sample types such as organism identifications.

Aspects of the systems and methods provided herein may be embodied in programming. Various aspects of the technology may be thought of as “products” or “articles of manufacture” typically in the form of executable code and/or associated data that is carried on or embodied in a type of machine readable medium. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

In some embodiments, the system is configured to communicate with one or more remote devices, such as a remote electronic. Such remote connection is facilitated using the communications interface. In some situations, the system presents information to (or requests information of actions from) the user by way of a user interface on an electronic device of the user (see below). The user interface can be a graphical user interface (GUI). In some cases, the GUI operates on an electronic device of the user, such as a portable electronic device (e.g., mobile phone, Smart phone). The electronic device can include an operating system for executing software and the graphical user interface of the electronic device.

In some embodiments, the system provides alerts, updates, notifications, warnings, and/or other communications to the user by way of a graphical user interface (GUI) operating on the system or an electronic device of the user. The GUI may permit the user to access the system to, for example, create or update a profile, view status updates, setup the system for sample preparation and processing, or view the results of sample preparation, processing and/or analysis. The system can be configured to operate only when a user provides indicia of permission, such as a key card and/or a password. The system can record and provide information on sample chain of custody, contamination or tampering. Systems to record and provide such information can include controls on access to operate the system (e.g., operator permission requirements); sample control (e.g., sensors to indicate introduction or removal of a sample from a cartridge); enclosure control (e.g., sensors indicating door opening and closing) and cartridge control (e.g, sensors for indicating insertion, proper seating and removal of cartridge).

In some embodiments, the system includes one or more modules for sample processing and/or analysis, and a controller for facilitating sample processing and/or analysis. The controller can include one or more processors, such as a central processing unit (CPU), multiple CPU's, or a multi-core CPU for executing machine-readable code for implementing sample processing and/or analysis. The system in some cases directs a sample sequentially from one module to another, such as from a sample preparation module to an electrophoresis module.

Although the foregoing invention has been described in some detail for purposes of clarity of understanding, it will be apparent that certain changes and modifications may be practiced within the scope of the invention. It should be noted that there are many alternative ways of implementing the processes and databases of the present invention. Accordingly, the present embodiments are to be considered as illustrative and not restrictive, and the invention is not to be limited to the details given herein.

What is claimed is:

1. A method of producing an electropherogram from raw electropherogram data comprising a sequence of one or more peaks, each peak comprising signal intensity values as a function of wavelength and time or position and each peak corresponding to one or more unique macromolecules, each macromolecule tagged with one of a plurality of different dyes, wherein each peak has a spectral contribution from one or more of the dyes, the method comprising:
(a) receiving the raw electropherogram data;
(b) for a first dye from plurality of different dyes, selecting from the raw electropherogram data one or more color peaks that contain signal intensity versus wavelength data for the first dye and substantially no signal intensity for any other dyes of the plurality of different dyes;
(c) determining, from the one or more color peaks identified in (b), a color spectrum of the first dye, wherein the color spectrum of the first dye comprises signal intensity values as a function of wavelength for only the first dye; and
(d) using the color spectrum of the first dye, together with color spectra of the other dyes of the plurality of different dyes, to deconvolve the raw electropherogram data to separate the contributions of each of the dyes to the raw electropherogram data and produce the electropherogram.

2. The method of claim 1, further comprising repeating operations (b)-(c) for at least one more of the other dyes of the plurality of different dyes.

3. The method of claim 1, further comprising repeating operations (b)-(c) for at least two more of the other dyes of the plurality of different dyes.

4. The method of claim 1, further comprising repeating operations (b)-(c) for each of the different dyes.

5. The method of claim 1, wherein the macromolecules are amplicons from amplification reactions of DNA sequences at two more loci of a genome or chromosome.

6. The method of claim 5, wherein the genome is a human genome.

7. The method of claim 5, wherein the loci are at polymorphism sites.

8. The method of claim 7, wherein the polymorphism sites are STR sites.

9. The method of claim 7, further comprising using the electropherogram to identify alleles of an individual who originated a sample that produced the raw electropherogram data.

10. The method of claim 7, wherein there are at least about sixteen loci, and at least three dyes.

11. The method of any preceding claim, further comprising performing electrophoresis on a sample comprising the macromolecules, wherein performing electrophoresis generates the raw electropherogram data.

12. The method of any preceding claim, wherein selecting one or more color peaks that contain signal intensity versus wavelength data for the first dye and substantially no signal intensity for any other dyes of the plurality of different dyes comprises: applying criteria for selecting one or more substantially isolated and substantially spectrally pure color peaks from the raw electropherogram data.

13. The method of claim 12, wherein the criteria comprise identifying color peaks having a portion that increases or decreases monotonically in a wavelength dimension, wherein positions on the wavelength dimension represent distinct wavelengths.

14. The method of claim 12, wherein the criteria comprise identifying color peaks having a portion that has a slope in a wavelength dimension of at least a predefined value, wherein positions on the wavelength dimension represent distinct wavelengths.

15. The method of claim 12, wherein the criteria comprise identifying peaks that are separated from other peaks by at least a threshold time duration or position difference.

16. The method of claim 12, wherein applying criteria for selecting one or more substantially isolated and substantially spectrally pure color peaks identifies multiple substantially isolated and substantially spectrally pure peaks.

17. The method of claim 16, further comprising combining the spectra of the multiple substantially isolated and substantially spectrally pure color peaks to produce the color spectrum of the first dye.

18. The method of claim 17, wherein combining the spectra the spectra of the multiple substantially isolated and substantially spectrally pure color peaks comprises producing a weighted average of the spectra of the multiple substantially isolated and substantially spectrally pure color peaks.

19. The method of claim 18, wherein producing a weighted average of the spectra of the multiple substantially isolated and substantially spectrally pure color peaks comprises weighting each of the spectra of the substantially isolated and substantially spectrally pure color peak according to its peak height and/or its peak width.

20. The method of claim 16, further comprising:
correlating the multiple substantially isolated and substantially spectrally pure color peaks to identify a subset of said multiple peaks that are more highly correlated than other of said multiple peaks that are not in the subset; and
combining the subset of substantially isolated and substantially spectrally pure peaks to produce the color spectrum of the first dye.

21. The method of any preceding claim, wherein the color data is provided in between fifty and five hundred distinct color channels.

22. The method of claim 21, wherein the signal intensity versus wavelength data for the color peaks was obtained using a spectrophotometer.

23. The method of any preceding claim, further comprising preparing a calibration matrix from the color spectrum of the first dye the other dyes of the plurality of different dyes and the other dyes of the plurality of different dyes, and wherein using the color spectrum of the first dye, together with color spectra of the other dyes of the plurality of different dyes to deconvolve the raw electropherogram data comprises applying the calibration matrix to the raw electropherogram data.

24. The method of claim 23, wherein the calibration matrix comprises color spectra of all the plurality of different dyes.

25. The method of any preceding claim, wherein a single sample is employed to produce the raw electropherogram data and the one or more color peaks that contain signal intensity versus wavelength data for the first dye and substantially no signal intensity for any other dyes of the plurality of different dyes.

26. The method of any preceding claim, wherein the macromolecules are oligonucleotides

27. The method of claim 1, wherein the number of unique macromolecules producing the raw electropherogram data is greater than the number of different dyes tagging the unique macromolecules.

28. The method of claim 1, further comprising using the electropherogram to identify a macromolecule corresponding to a peak in the raw electropherogram data.

29. A system comprising:
a capillary tube arranged to receive a sample comprising a plurality of unique macromolecules and run the sample through the capillary tube so that different ones of the unique macromolecules pass through an interrogation region of the capillary tube at different times;
optical elements arranged with respect to one another to receive color signals from the interrogation region; and
a controller designed or configured to:
(i) convert the color signals into raw electropherogram data comprising a sequence of peaks, each peak comprising signal intensity values as a function of wavelength and time or position and each peak corresponding to one or more unique macromolecules, each macromolecule tagged with one of a plurality of different dyes, wherein each peak has a spectral contribution from one or more of the dyes,
(ii) for a first dye from plurality of different dyes, select from the raw electropherogram data one or more color peaks that contain signal intensity versus wavelength data for the first dye and substantially no signal intensity for any other dyes of the plurality of different dyes,
(iii) determine, from the one or more color peaks identified in (ii), a color spectrum of the first dye, wherein the color spectrum of the first dye comprises signal intensity values as a function of wavelength for only the first dye, and
(iv) use the color spectrum of the first dye, together with color spectra of the other dyes of the plurality of different dyes, to deconvolve the raw electropherogram data to separate the contributions of each of the dyes to the raw electropherogram data and produce the electropherogram.

30. The system of claim 29, wherein the controller is further designed or configured to perform or cause to be performed electrophoresis on a sample comprising the macromolecules, wherein performing electrophoresis generates the raw electropherogram data.

31. The system of claim 29, wherein the controller is further designed or configured to perform or cause to be performed the operations of any of claims 2-4, 9, 12-20, 23, 24, and 28.

32. A method of analyzing a sample comprising one or more unique macromolecules tagged with one of a plurality of different dyes, the method comprising:
performing an electrophoresis run on the sample to produce first raw electropherogram data comprising a sequence of peaks, each corresponding to one or more of the unique macromolecules, wherein each peak has a spectral contribution from one or more of the plurality of different dyes;
analyzing the first raw electropherogram data and identifying an uncalibrated dye, from among the plurality of different dyes associated with the macromolecules, for which a substantially pure spectrum is not identified from the raw electropherogram data;
identifying a substantially pure spectrum of the uncalibrated dye from second raw electropherogram data of a related electrophoresis run; and
using the substantially pure spectrum of the uncalibrated dye, from the second raw electropherogram data, to deconvolve the first raw electropherogram data to separate the contributions of each of the plurality of different dyes to the first raw electropherogram data to thereby produce a first electropherogram.

33. The method of claim 32, further comprising: from the first raw electropherogram data, extracting multi-channel color data as a function of time or position, wherein the color data represents the spectral contributions from the plurality of different dyes.

34. The method of claim 33, wherein the related electrophoresis run is a next sequential electrophoresis run on the same apparatus as used to produce the first raw electropherogram data.

35. The method of claim 32, 33, or 34, wherein the first raw electropherogram data and the second raw electropherogram data are produced using runs conducted at the same position in a single apparatus.

36. The method of any of claim 32 or 33, wherein the first raw electropherogram data and the second raw electropherogram data are produced using runs conducted at two different positions at the same time in a single apparatus.

37. The method of any of claims 32-36, further comprising, prior to deconvolving the first raw electropherogram data, scaling the substantially pure spectrum of the uncalibrated dye, from the second raw electropherogram data.

38. The method of claim 37, wherein the scaling comprises modifying the substantially pure spectrum of the uncalibrated dye using information obtained about the spectra of a first calibrated dye obtained using both the first raw electropherogram data and the second raw electropherogram data.

39. The method of claim 32, wherein the number of unique macromolecules is greater than the number of different dyes.

40. The method of claim 32, wherein each peak of the first raw electropherogram data comprises signal intensity values as a function of wavelength and time or position.

41. A system comprising:
a capillary tube arranged to receive a sample comprising a plurality of unique macromolecules and run the sample through the capillary tube so that different ones of the unique macromolecules pass through an interrogation region of the capillary tube at different times;
optical elements arranged with respect to one another to receive color signals from the interrogation region; and
a controller designed or configured to:
(i) convert the color signals into raw electropherogram data comprising a sequence of peaks, each corresponding to one or more of the plurality of unique macromolecules tagged with one of a plurality of different dyes,
(ii) perform an electrophoresis run on the sample to produce first raw electropherogram data comprising a sequence of peaks, each corresponding to one or more of the unique macromolecules, wherein each peak has a spectral contribution from one or more of the plurality of different dyes,
(iii) analyze the first raw electropherogram data and identifying an uncalibrated dye, from among the plurality of different dyes associated with the macromolecules, for which a substantially pure spectrum is not identified from the raw electropherogram data,
(iv) identify a substantially pure spectrum of the uncalibrated dye from second raw electropherogram data of a related electrophoresis run, and
(v) use the substantially pure spectrum of the uncalibrated dye, from the second raw electropherogram data, to deconvolve the first raw electropherogram data to separate the contributions of each of the plurality of different dyes to the first raw electropherogram data to thereby produce a first electropherogram.

42. The system of claim 41, wherein the controller is further designed or configured to perform or cause to be performed electrophoresis on a sample comprising the macromolecules, wherein performing electrophoresis generates the raw electropherogram data.

43. The system of claim 41, wherein the controller is further designed or configured to perform or cause to be performed the operations of any of claims 33, 37, and 38.
